(19) United States
(12) Patent Application Publication
(10) Pub. No.: US 2015/0238150 A1
(43) Pub. Date: Aug. 27, 2015

(54) SMARTWATCH WITH A MULTI-PURPOSE SENSOR FOR REMOTE MONITORING OF A PATENT

(71) Applicant: Gestln Time Inc., Palo Alto, CA (US)

(72) Inventor: Suresh Subramaniam, Palo Alto, CA (US)

(73) Assignee: Gestln Time Inc., Palo Alto, CA (US)

(21) Appl. No.: 14/189,877

(22) Filed: Feb. 25, 2014

(51) Int. Cl. A61B 5/00; G08B 21/04; H04Q 9/00

(52) U.S. Cl. CPC A61B 5/74; H04Q 9/00; G08B 21/0446

(57) ABSTRACT

A method and apparatus for remote monitoring of a medical patient using a smartwatch is described. A sensor device is placed in physical contact with the patient. The sensor communicates with a smartwatch using Bluetooth or another wireless communication protocol. The smartwatch then communicates with a remote server that compiles data regarding the patient. The system can gather data such as the patient's temperature, the amount of exercise undertaken by the patient, the amount of sleep by the patient, and whether the patient has physically fallen.

### PRIORITY CLAIM

This application claims priority under 35 U.S.C. Section 120 and is a continuation-in-part of U.S. patent application Ser. No. 13/962,860, filed on Aug. 8, 2013 and titled “Method and Apparatus for Integrated Medical Services Using a Multi-Purpose Sensor for Remote Monitoring of a Patient,” which is a continuation-in-part of U.S. patent application Ser. No. 13/842,191, filed on Mar. 15, 2013 and titled “Method and Apparatus for Providing Integrated Medical Services,” which is incorporated by reference.

### TECHNICAL FIELD

A smartwatch for remote monitoring of a medical patient is described. A sensor device is placed in physical contact with the patient through a smartwatch. The sensor communicates with a computing device using Bluetooth or another wireless communication protocol. The computing device then communicates with a remote server that compiles data regarding the patient. The system can gather data such as the patient's temperature, the amount of exercise undertaken by the patient, the amount of sleep by the patient, and whether the patient has physically fallen.

### BACKGROUND OF THE INVENTION

The collection and analysis of medical data in the prior art is relatively localized. Patients typically visit their physicians, and the physicians collect and record data (such as blood pressure) at the physician's office. Patients are able to collect certain data at home, such as by using blood pressure devices that can be purchased at a pharmacy, but the prior art does not include any satisfactory mechanism for integrating the collection of medical data at the home and the physician's office. In addition, there is no automated way to share medical data collected at the home with your physician.

Texas Instruments (TI) recently introduced the CC2541 SensorTag device. The SensorTag device is an example of a remote sensor 700, represented in FIG. 15, where a functional depiction of remote sensor 700 is shown. Remote sensor 700 is designed to communicate with other devices, such as smartphones, using a Bluetooth transceiver 610. Remote sensor 700 comprises infrared temperature sensor 620, humidity sensor 630, pressure sensor 640, accelerometer 650, gyroscope 660, and magnetometer 670. Remote sensor 700 also comprises button 680 and button 690.

When placed on or in close proximity to a human patient, infrared temperature sensor 620 can determine the temperature of the human patient. Humidity sensor 630 can measure the humidity of the environment surrounding remote sensor 700. Pressure sensor 640 determine the pressure generated by the remote sensor 700 being in physical contact with the human patient. Accelerometer 650 can measure acceleration of remote sensor 700 in three directions (e.g., the X, Y, and Z directions). Gyroscope 660 can determine the movement of remote sensor 700 in three directions (e.g., the X, Y, and Z directions). Magnetometer 670 is a compass. Button 680 and button 690 each can be pressed by the human patient. When this happens, remote sensor 700 generates a signal indicating which button—button 680 or button 690 or both—was pressed. Other design aspects of remote sensor 700 are described in the “CC2541 SensorTag Quick Start Guide,” which is submitted with this application and is incorporated by reference. To date, the development of applications for using remote sensor 700 is in its infancy.

In another area of the prior art, smartwatches are known. A smartwatch is a watch with traditional time-keeping functionality as well as processing capability and wireless connectivity.

What is needed is a smartwatch housing remote sensor 700 for protecting remote sensor 700 and for attaching it to a human patient. What is further needed is a backend application that gathers information from remote sensor 700, processes the information, and generates alerts and reports based upon pre-determined criteria.

What is further needed is an integrated approach to medical data collection and analysis, including use of data from remote sensor 700, and an improved medium by which patients and physicians can communicate.

### SUMMARY OF THE INVENTION

The aforementioned problem and needs are addressed through an embodiment for gathering patient data using remote sensor 700, transferring that data to a computing device, uploading the data into a server, processing the data, generating alerts to a physician if necessary, and providing portals by which the patient and physician can communicate and exchange data and information. Also disclosed are different enclosures for remote sensor 700.

### BRIEF DESCRIPTION OF THE DRAWINGS

FIG. 1 depicts an embodiment of a system for providing integrated medical services.

FIG. 2 depicts software components of a server for providing integrated medical services.

FIG. 3 depicts hardware components of an embodiment of a server.

FIG. 4 depicts an exemplary patient portal login page.

FIG. 5A depicts an exemplary patient portal welcome page.

FIG. 5B depicts an exemplary patient portal welcome page.

FIG. 6 depicts an exemplary patient portal messages page.

FIG. 7 depicts an exemplary patient portal learning page.

FIG. 8 depicts an exemplary patient portal account settings page.

FIG. 9 depicts an exemplary patient portal journal page.

FIG. 10 depicts an exemplary physician portal login page.

FIG. 11 depicts an exemplary physician portal welcome page.

FIG. 12 depicts an exemplary physician portal selected patient page.

FIG. 13 depicts an embodiment of a computing device with various display options.

FIG. 14 depicts an embodiment of display eyewear.

FIG. 15 depicts a functional overview of a prior art remote sensor.

FIG. 16 depicts an embodiment of a monitoring device comprising a remote sensor.

FIG. 17 depicts an embodiment of a method of remote monitoring.

FIG. 18 depicts another embodiment of a method of remote monitoring.

FIG. 19 depicts another embodiment of a method of remote monitoring.

FIG. 20 depicts another embodiment of a method of remote monitoring.

FIG. 21 depicts an embodiment of a smartwatch containing a remote sensor.

FIG. 22 depicts hardware components of a smartwatch containing a remote sensor.

FIG. 23 depicts a smartwatch comprising a heartrate monitor.

FIG. 24 depicts a smartwatch comprising a speaker, microphone, and audio port.

FIG. 25 depicts a smartwatch acting as a gateway for a device.

### DETAILED DESCRIPTION OF THE PREFERRED EMBODIMENTS

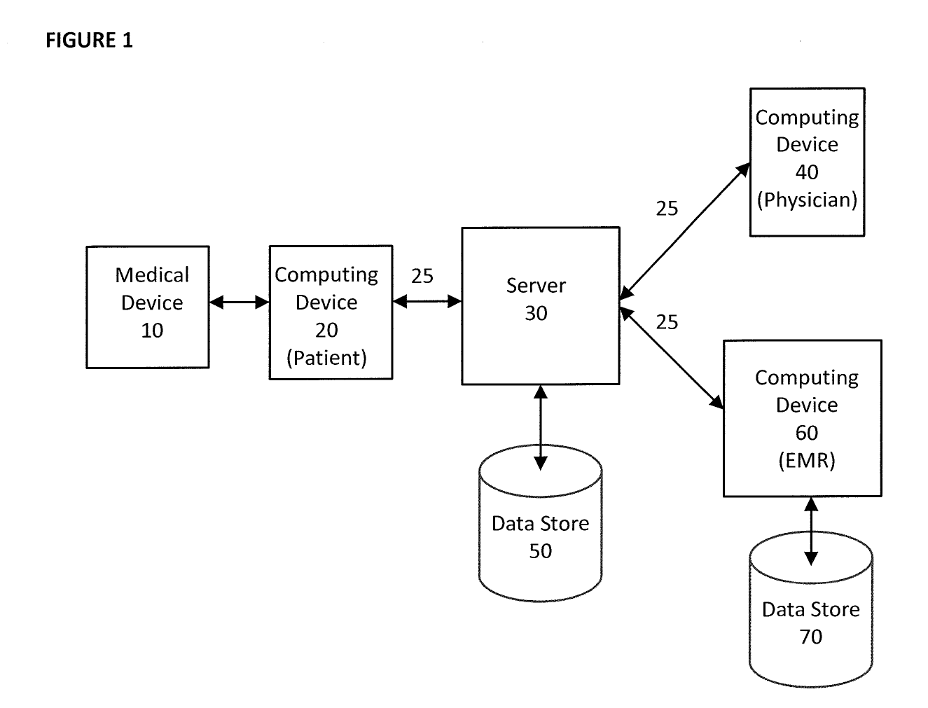

An embodiment will now be described with reference to FIG. 1. Medical device 10 collects data from a patient in the home, physician's office, or any other site. Medical device 10 can be a device to measure weight, heart rate, blood pressure, blood sugar levels, contractions, fetal heart rate, or any other device to take a measurement relevant to the health of the patient. The data is transmitted to computing device 20 over known interfaces, such as a USB connector, Bluetooth, or Wifi (e.g., 802.11).

Computing device 20 can be a desktop, notebook, server, mobile phone, tablet, game console, or any other type of device with a processor, memory, and network interface. Computing device 20 communicates with server 30 over a network 25. Network 25 optionally can be the Internet and can be hardwired, wireless, or some combination of the two. In this embodiment, computing device 20 is operated by a patient.

Server 30 is coupled to data store 50. Server 30 is also coupled to computing device 40 over network 25. Computing device 40 can be a desktop, notebook, server, mobile phone, tablet, game console, or any other type of device with a processor, memory, and network interface. In this embodiment, computing device 40 is operated by a physician.

Server 30 is coupled to computing device 60 over network 25. Computing device 60 can be a desktop, notebook, server, mobile phone, tablet, game console, or any other type of device with a processor, memory, and network interface. Computing device 60 is coupled to data store 70. In this embodiment, computing device 60 is operated by an electronic medical records (EMR) company, such as a health insurance company or billing agency.

Data store 50 and data store 70 optionally can each be a relational database for storing data records, such as a MySQL database.

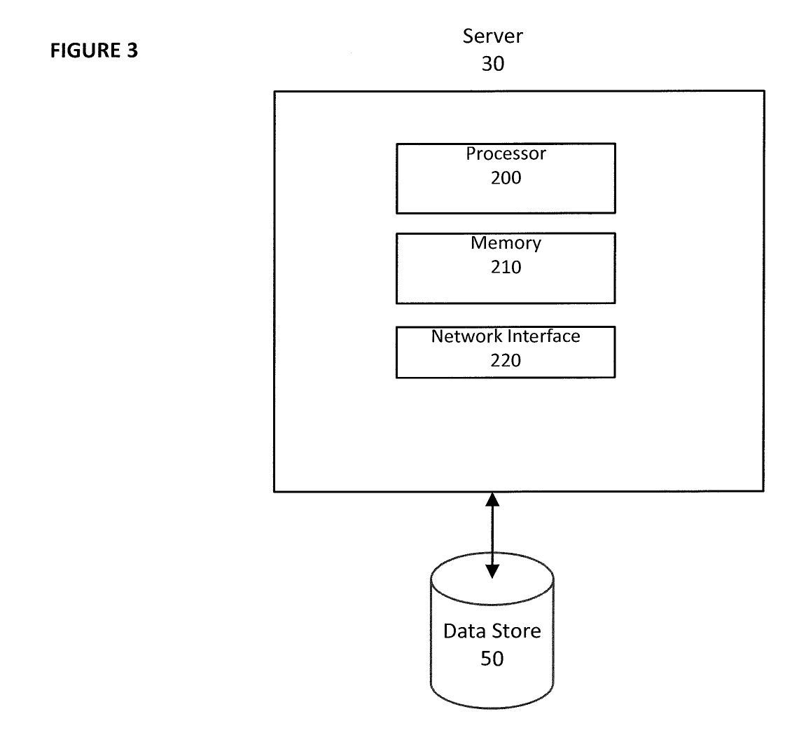

With reference to FIG. 2, various software components of server 30 are depicted. Server 30 comprises risk classification engine 100, recommendation engine 110, notification engine 120, messaging engine 130, web server 140, application interface 150, and integration gateway 160.

Risk classification engine 100 comprises lines of code executed by processor 200 of server 30. Risk classification engine 100 analyzes the data collected from a patient using medical device 10, compares it against known criteria, such as those available from the American Congress of Obstetricians and Gynecologists (ACOG), and characterizes the patient in her current state with risk ratings, such as “low risk,” “medium risk,” or “high risk.” For example, a patient with an extremely high blood sugar reading could be categorized as “high risk.” The risk ratings are stored in data store 50 and are associated with the patient's profile.

Recommendation engine 100 comprises lines of code executed by processor 200 of server 30. Recommendation engine 100 generates a recommendation based on the data collected from the patient and existing data in data store 50 and makes a recommendation for a physician. For example, for a “high risk” patient, the recommendation could be “Call patient immediately,” or “Instruct patient to engage in 30 minutes of exercise to lower blood sugar level.” The recommendation is stored in data store 50 and also made known to notification engine 120 and/or messaging engine 130.

Notification engine 120 comprises lines of code executed by processor 200 of server 30. Notification engine 120 provides notifications or alerts to patients, physicians, or other persons using email, SMS or MMS messages, intra-system messages, phone calls, or on-screen alerts. For example, if recommendation engine 100 generated a recommendation of “Call patient immediately,” notification engine 120 would send that message to the physician associated with the patient in data store 50.

Messaging engine 130 comprises lines of code executed by processor 200 of server 30. It permits messages to be sent among patients, physicians, and server 30.

Web server 140 comprises lines of code executed by processor 200 of server 30. Web server 140 generates a web-based portal for patients and a web-based portal for physicians, as described in subsequent paragraphs.

Web server 140 is capable of generating web pages that are specifically suited for the particular computing device 20 or 40 that is being used. For example, different cascading style sheets can be used for a desktop computer and a mobile device, such that the web pages are optimized for display on the particular device. The underlying device can be identified, and the appropriate cascading style sheet selected, using well-known HTTP communication.

Application interface 150 comprises lines of code executed by processor 200 of server. Application interface 150 is capable of interfacing with applications running on computing device 20, computing device 40, or computing device 60 using Application Programming Interfaces (APIs), such as APIs known and used in conjunction with the iOS and Android operating systems, facebook, and Twitter.

Integration gateway 160 comprises lines of code executed by processor 200 of server 30. Integration gateway 160 interfaces with computing device 60 to exchange data relating to EMR. Computing device 60 optionally can be operated by a health insurance provider. Integration gateway 160 is designed to exchange data in a protocol and/or format that is expected or specified by the computing device 60 or its operator. For example, health insurance companies typically require that data be sent to them in a certain format with certain billing codes.

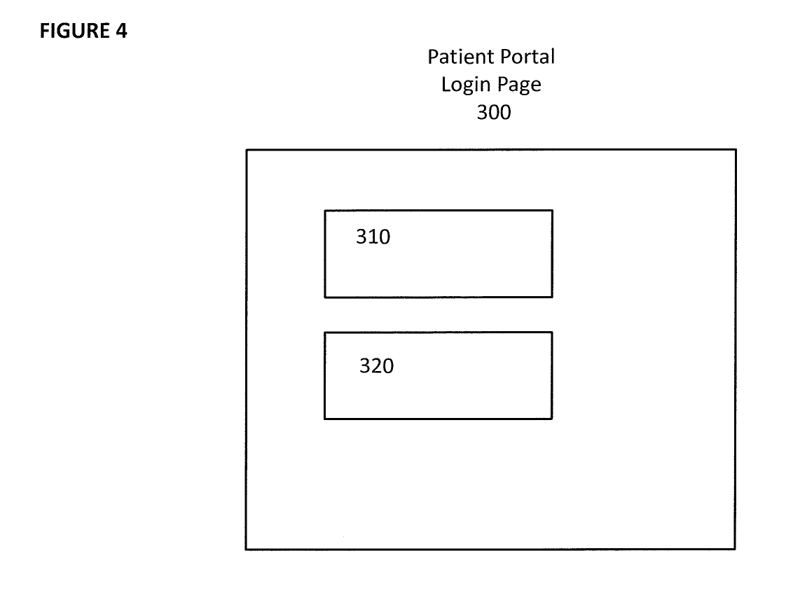

FIG. 3 depicts server 30 and some of its hardware components. Server 30 comprises a processor 200, memory 210, and network interface 220. Network interface 220 can comprise one or more wired or wireless interfaces, such as an Ethernet interface, WiFi (e.g., 802.11), WiMax, cell phone (e.g., 3G or 4G), Bluetooth, or other interface.

Patient Portal

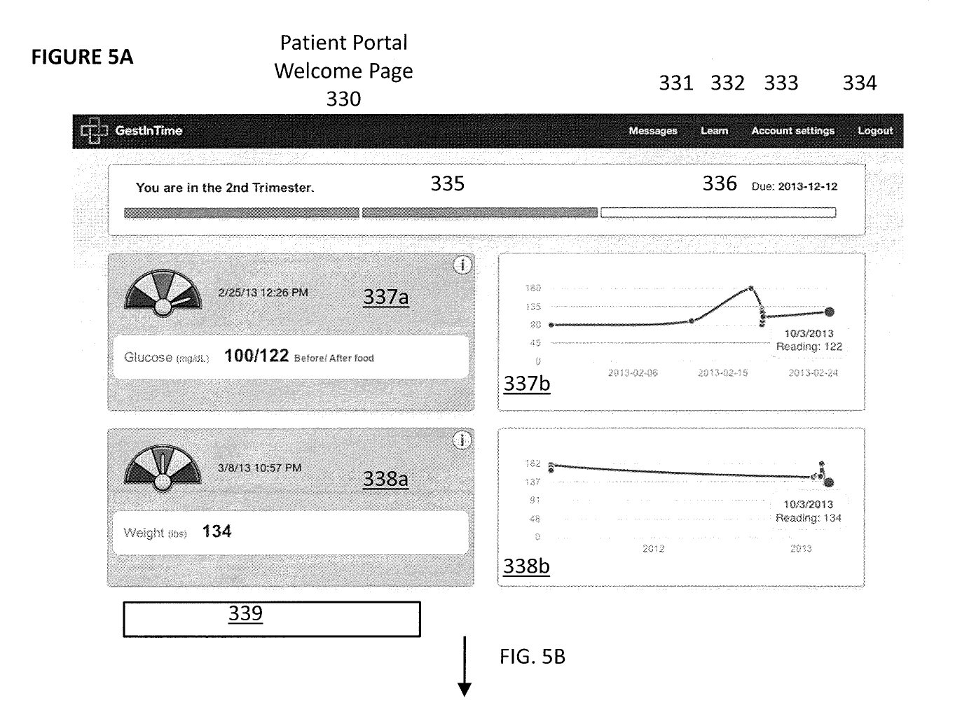

The patient portal accessible and used by a patient will now be described. FIG. 4 shows a screenshot of an exemplary login page 300 for patients. Login page 300 is generated by server 30 and is sent to computing device 20 using web server 140 (if served as a web page) or application interface 150 (if served as a non-web browser application). Login page 300 comprises input device 310 to receive the patient's user name and input device 320 to receive the patient's password. Input device 310 and input device 320 can comprise, for example, HTML text boxes.

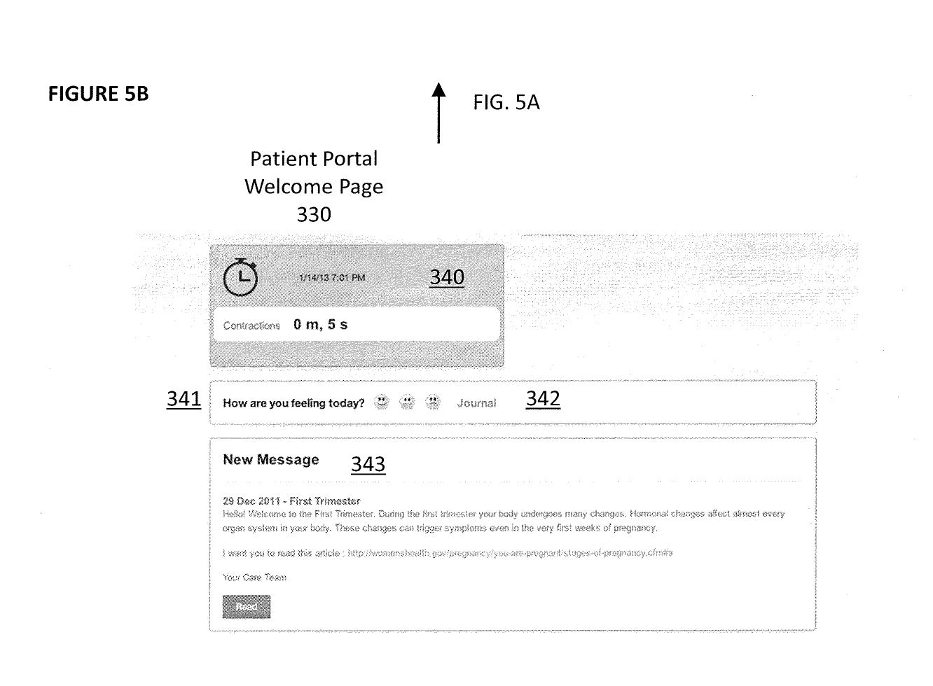

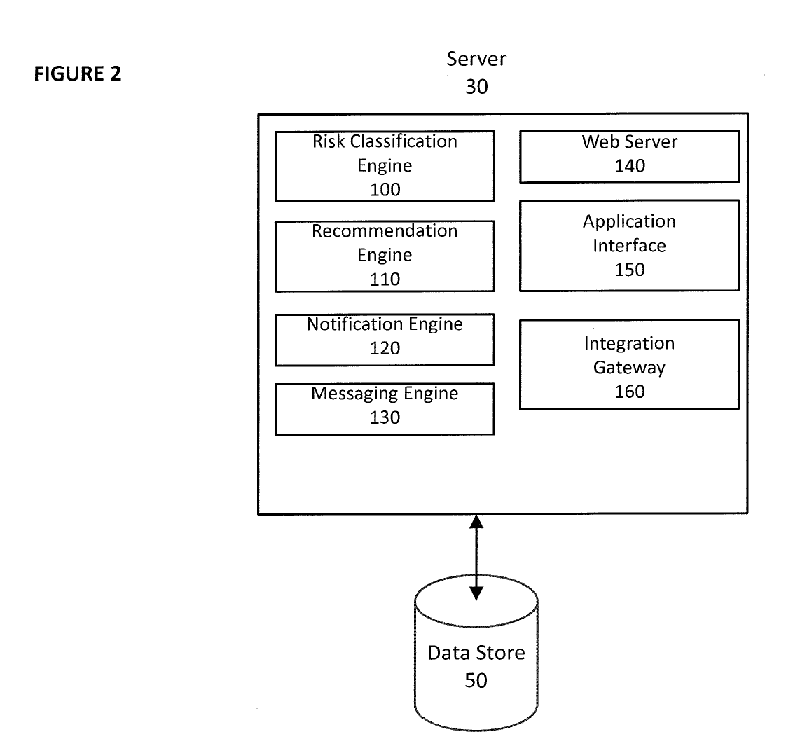

FIGS. 5A and 5B show screenshots of an exemplary “welcome page” 330 for a particular patient. This page might be displayed, for example, after a patient logs in using the login screen depicted in FIG. 4. Welcome page 300 comprises user input device 331 (to access messages), input device 332 (to access a learning page), input device 333 (to access account settings) and input device 334 (to logout of the server). Input devices 331, 332, 333, and 334 can be, for example, HTML buttons, tabs, or links.

Welcome page 330 comprises graphical timeline 335 that can show major health events for the patient. For example, graphical timeline 335, for a maternity patient, might show the number of trimesters that have elapsed, the temporal position within the current trimester, and major tests and events that have occurred (such as an ultrasound test). Welcome page 330 also contains an entry 336 displaying the patient's due date or upcoming appointment dates.

Welcome page 330 further comprises data box 337a, graph 337b, data box 338a, and graph 338b. Data boxes 337a, 338b, and 340 display important data regarding the patient's health, such as current or recent readings of important metrics, such as blood sugar level, weight, blood pressure, or heart rate. Graphs 337b and 338b display the data in graphical form. Optionally, graphs 337b and 338b can be displayed in animated form to show how the data has changed over time. Optionally, data boxes 3371, 338b, and 340 and graphs 337b and 338b can compare the patient's data against ideal or expected data for those items or against the patient's prior data (for example, from a previous pregnancy), or against data collected from other persons such as persons within the patient's social network.

Welcome page 330 further comprises data upload field 339. Data upload field 339 comprises a button, menu, link, or other input device that, when selected by a patient, will provide an interface by which the patient can upload or enter data from a medical device 10. For example, data upload field 339 can generate a new page with a text box and menu item to enable a patient to type in data and to indicate the type of data it is (e.g., 150 lbs, weight). Data upload field 339 also can enable computing device 20 to receive data directly from medical device 10.

Welcome page 330 further comprises input field 341 to receive information regarding the patient's current mood. In this embodiment, the patient can click on one of three icons to convey her mood (happy; sad; or in between). A patient can create a journal entry or access prior journal entries by selecting input field 342.

Welcome page 330 further comprises new messages field 343 to display any new or recent messages received from a physician or elsewhere.

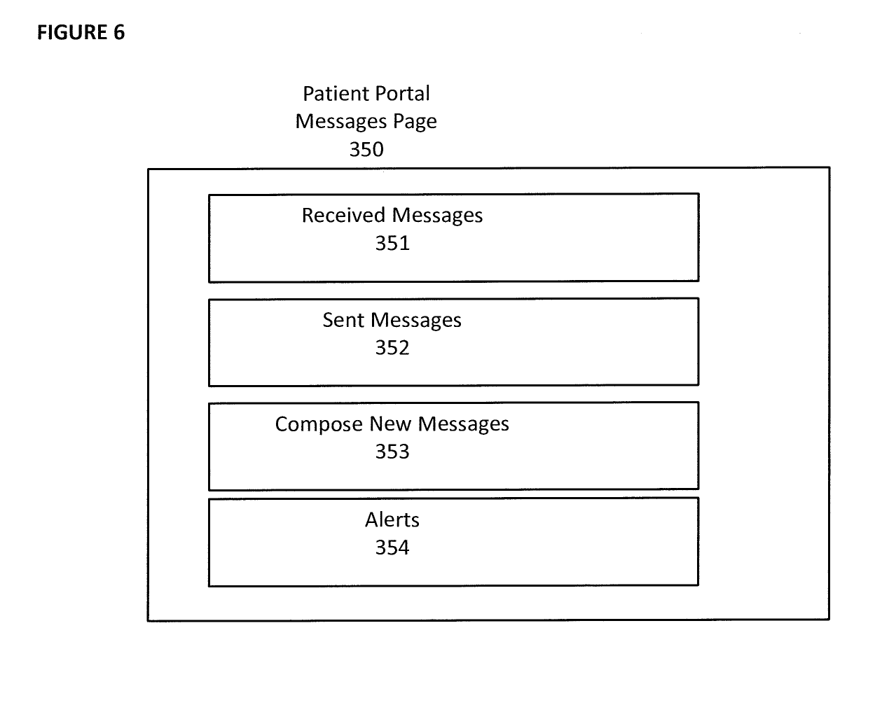

FIG. 6 depicts exemplary patient portal messages page 350, such as may be accessed through input device 331 on welcome page 330. Patient portal messages page 350 comprises field 351 to display received messages; field 352 to display sent messages, field 353 to allow the patient to composes a new message; and field 354 to display any alerts generated by server 30.

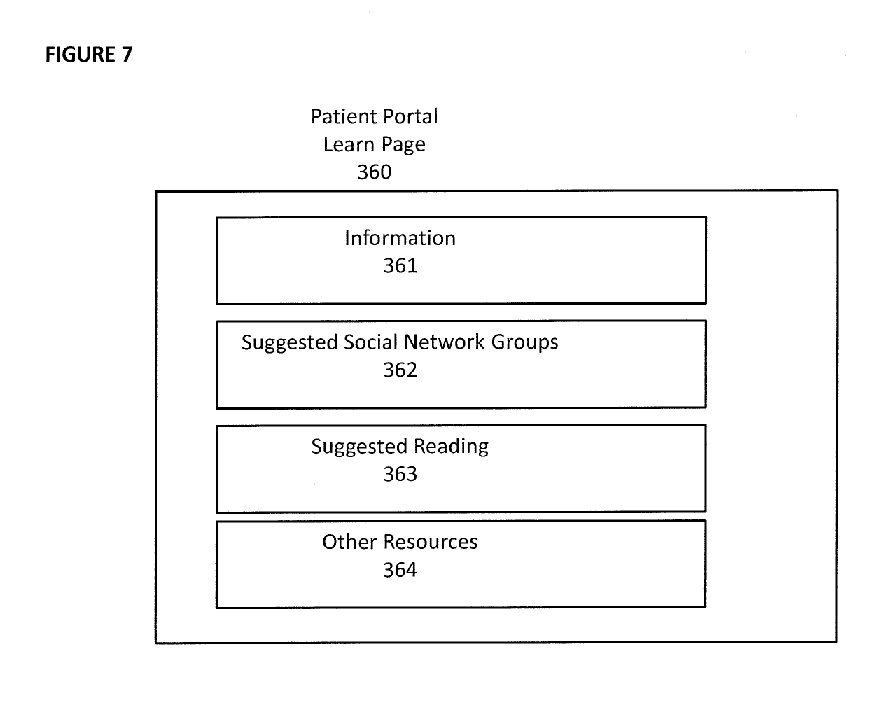

FIG. 7 depicts exemplary patient portal learning page 360, such as may be accessed through input device 332 on welcome page 330. Patient portal learning page 360 comprises field 351 to display information, field 362 to suggest social network groups for the patient (based on recommendations from recommendation engine 110), field 363 to provide suggested reading materials (based on recommendations from recommendation engine 110), and field 364 to display other resources for the patient (such as videos).

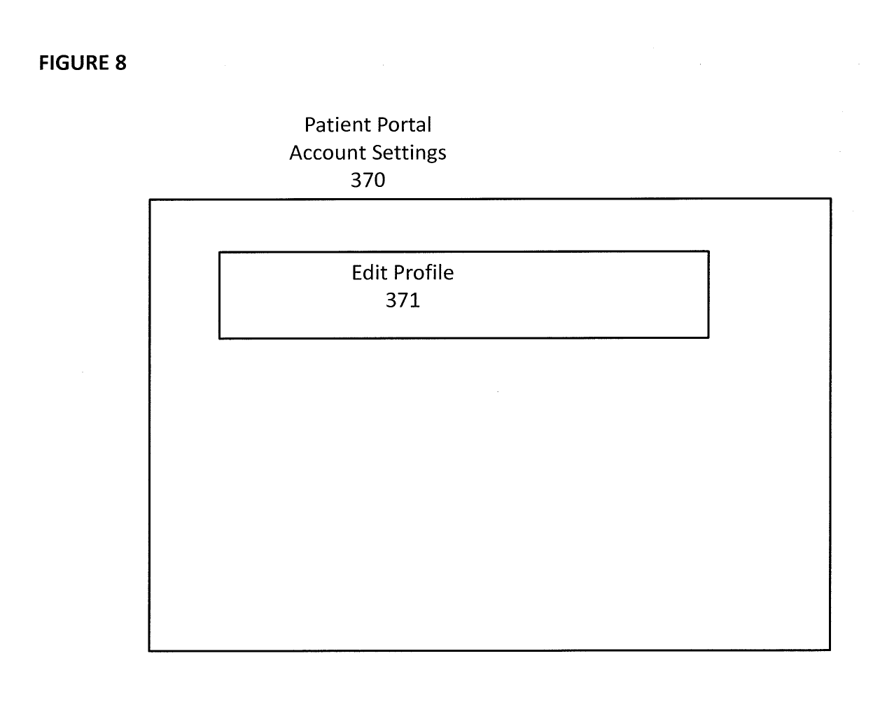

FIG. 8 depicts exemplary patient portal account settings page 370, such as may be accessed through input device 333 on welcome page 330. Patient portal account settings page 360 comprises field 371 to allow the patient to edit his or her profile, such as by editing information concerning name, address, medical plan, demographics, etc.

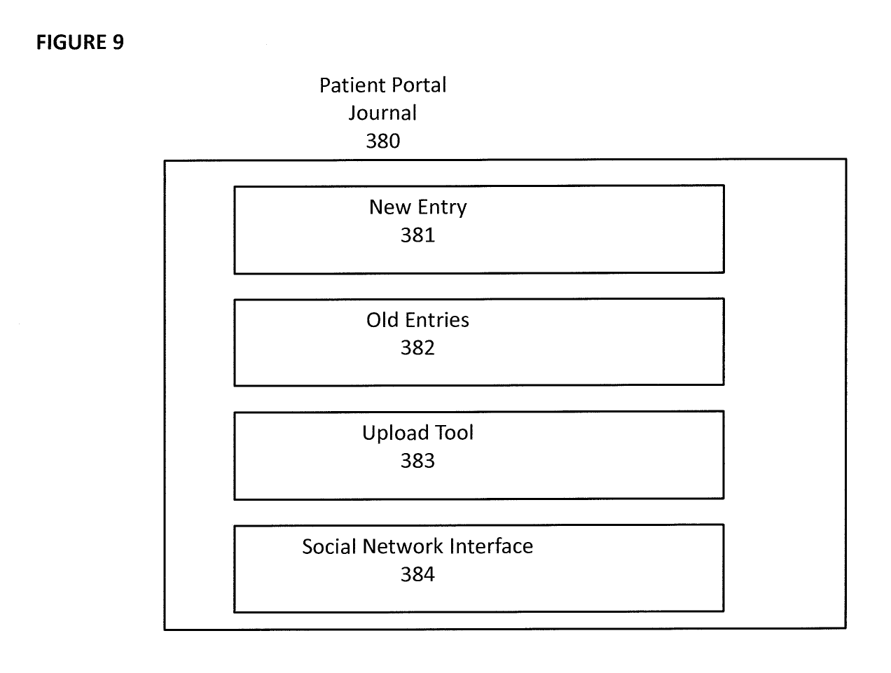

FIG. 9 depicts exemplary patient portal journal page 380, such as may be accessed through input device 342 on welcome page 330. Patient portal account journal page 380 includes field 381 for creating a new journal entry, field 382 for reviewing old journal entries, field 383 for uploading data or files (such as photos), and social network interface 384 for sharing journal entries for uploaded data or files (such as a “post on facebook” button).

Physician Portal

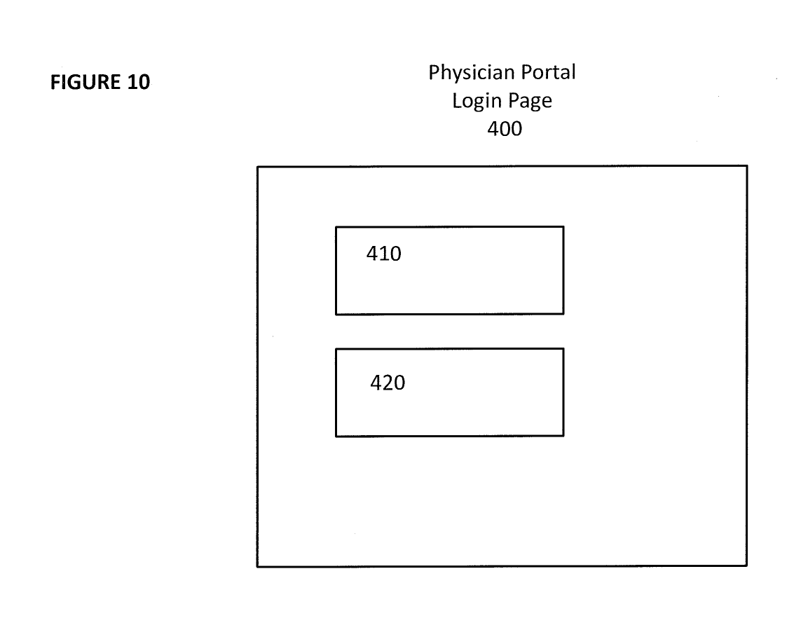

The physician portal accessible and used by a physician will now be described. FIG. 10 shows a screenshot of an exemplary login page 400 for patients. Login page 400 is generated by server 30 and is sent to computing device 40 using web server 140 (if served as a web page) or application interface 150 (if served as a non-web browser application). Login page 400 comprises input device 410 to receive the patient's user name and input device 420 to receive the patient's password. Input device 410 and input device 420 can comprise, for example, HTML text boxes.

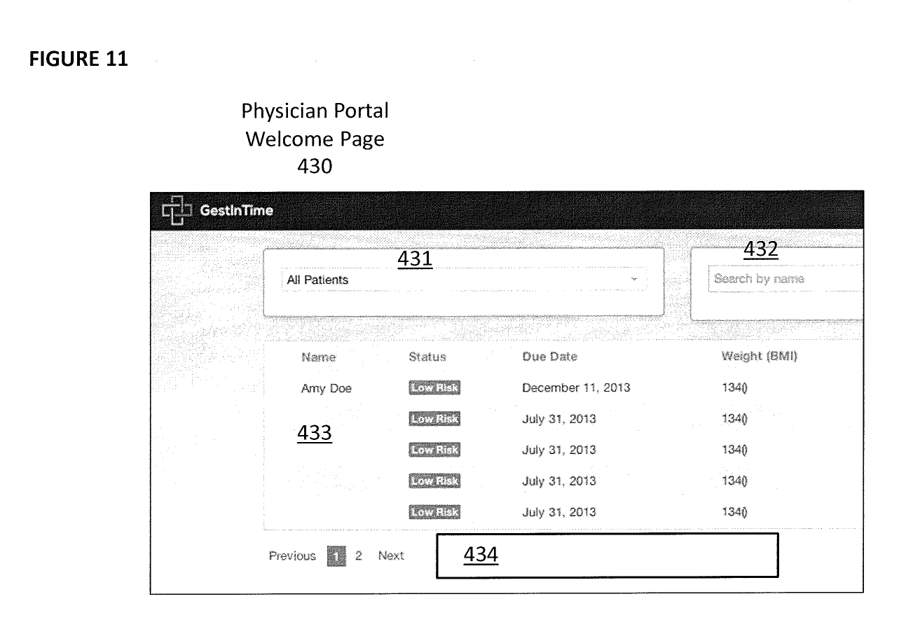

FIG. 11 shows a screenshot of an exemplary “welcome page” 430 for a particular physician. This page might be displayed, for example, after a physician logs in using the login screen depicted in FIG. 10. Welcome page 430 comprises user field 431 for selecting “all patients” or a particular patient. User field 341 might include a drop-down menu, pop-up menu, scroll entries, etc. Welcome page 430 further comprises text box 432 to enable a physician to search by name (which the physician types into the box). Welcome page 430 further comprises field 433 for patients, displaying key information for each patient (such as risk categorization, due date, and weight). Welcome page 430 further comprises text box 434 for displaying alerts, notifications, or anything else warranting the physician's immediate attention. For example, text box 434 can display notifications from notification engine 120 (“Call patient immediately”).

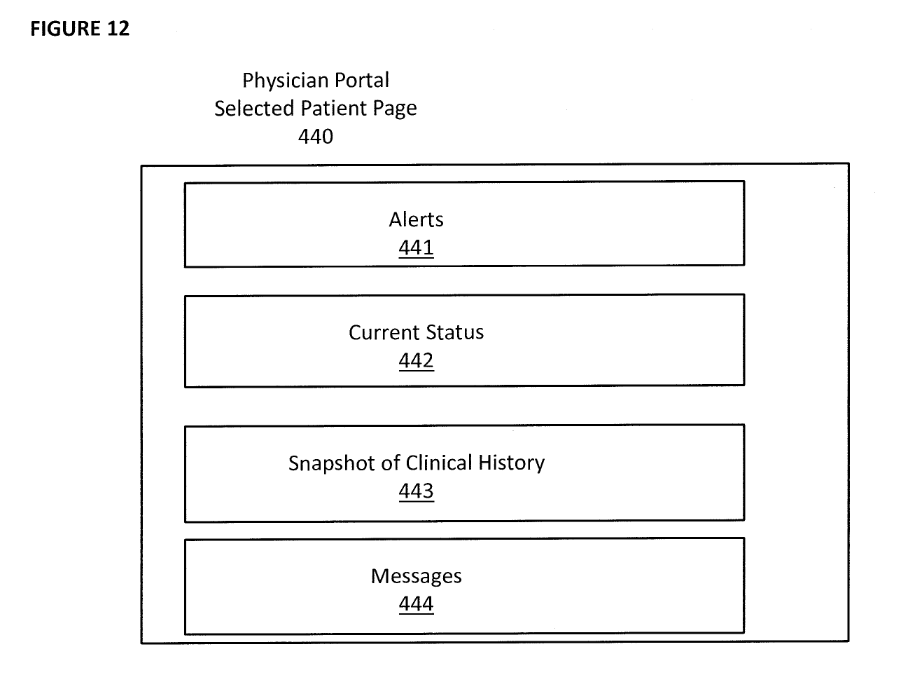

FIG. 12 depicts exemplary physician portal selected patient page 440. This page might be generated, for example, when the patient selects a particular patient on welcome page 430. Physician portal selected patient page 440 comprises field 441 to display alerts or notifications concerning that patient, field 442 to display the current status of the patient, field 443 for displaying a snapshot of the patient's clinical history, and field 444 for displaying any messages from or concerning that patient.

Display Options

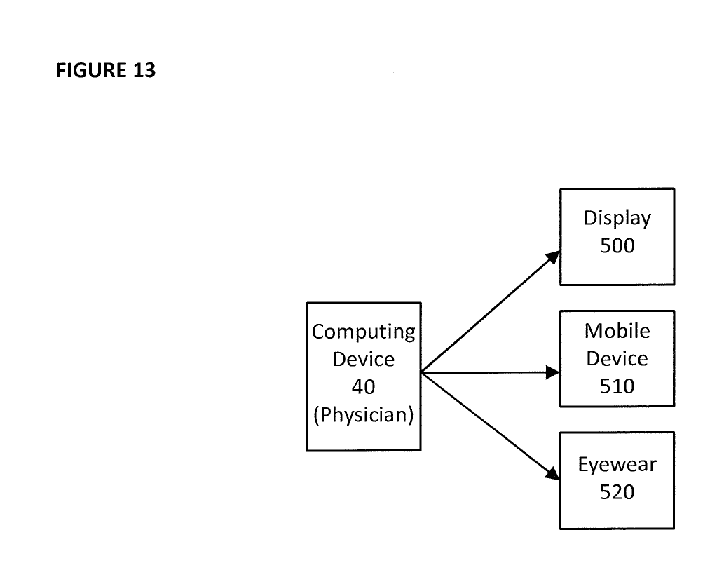

FIG. 13 depicts computing device 40 and various mechanisms for a physician to view the physician portal or relevant pages therein. These mechanisms include a display 500 (such as an LCD), mobile device 510 (such as a tablet or mobile phone), and eyewear 520.

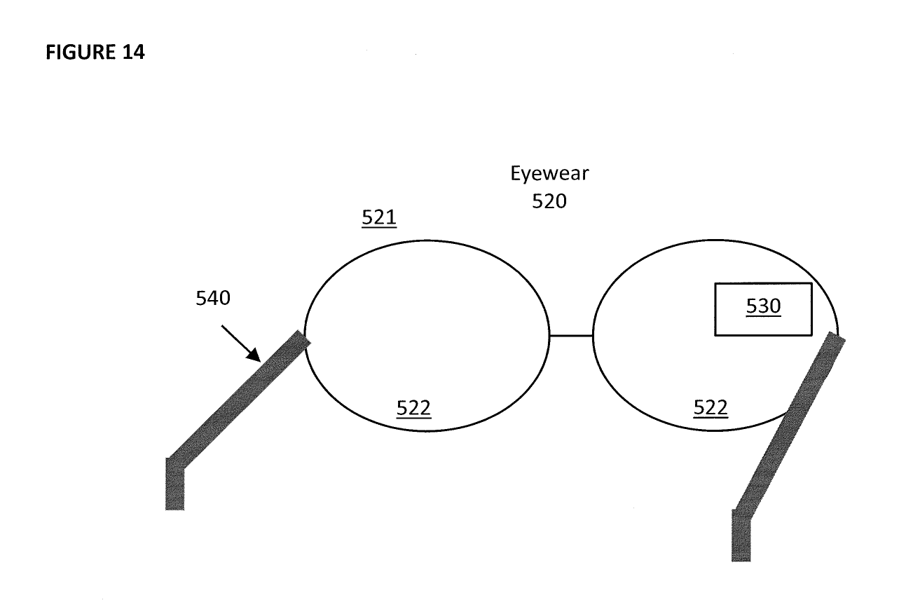

FIG. 14 depicts an example of eyewear 520. Eyewear 520 comprises lenses 522 and frame 521 (just as with normal glasses). But it also includes display unit 530 and processing and transmission unit 540 (embedded within the frame 521).

An example of eyewear 520 was recently announced by Google as the “Google Glass” product. Eyewear 520, such as the Google Glass, comprises a display unit 530 that displays data that you could otherwise display on an LCD or other display. For example, all of the pages described previously for the physician portal could be displayed on display unit 530.

The possible uses of eyewear 520 by physicians are numerous. For example, a physician could: (a) view pages from the physician portal on display unit 530 during a patient examination, during a remote consultation, or during a collaborative session with a fellow physician (e.g., two physicians viewing the same x-ray); (b) look at the patient in the physician's office while the display unit 530 displays patient medical data such as blood pressure, etc.; (c) apply physical pressure to the patient and get instant visual feed-back on soreness, pain points, soft-tissue, broken bones etc. The physician's view can be augmented with enhanced clinical data, such as heart rate; (d) look at a patient's hospital ID band (which gets scanned) and then view the patient's information on display unit 530; (e) Look at the patient and then have server 30 perform image/facial recognition to identify the patient and access and display his or her information in display unit 530; and (f) examine patient, and can get assisted by visual feedback critical information such as simulated images of the patient's internal organs. For example, the physician would be able to identify the location of the patient's spleen and then feel the spleen, because he or she could see the exact location of the spleen as a visual overlay of the patient's internal organ structure on the patient.

An embodiment will now be described with reference to FIG. 3. Medical device 10 is the same prior art device described previously with reference to FIG. 1. The output of medical device 10 is provided as an input to processing device 40. In this particular example, the output is EKG data, but the same principles apply to any periodic data collected from a medical device.

In one embodiment, processing device 40 is a computing device (such as a desktop, notebook, server, tablet, mobile device, or other computer) comprising a processor, memory, non-volatile storage (such as a hard disk drive or flash memory array), I/O connection (such as a USB connection) for communicating with a medical device, and an I/O connection for sending output to a display, printer, or other device. Optionally, processing device 40 can itself include a display (as might be the case if processing device 40 is a tablet or mobile device). Processing device 40 comprises software code for performing the functions described herein.

Remote Monitoring of Patient

In one embodiment, medical device 10 can be remote sensor 700. Remote sensor 700 must be placed in physical contact with the patient for it to collect data as intended. At the same time, it is important to protect remote sensor 700 from the physical elements and physical contact.

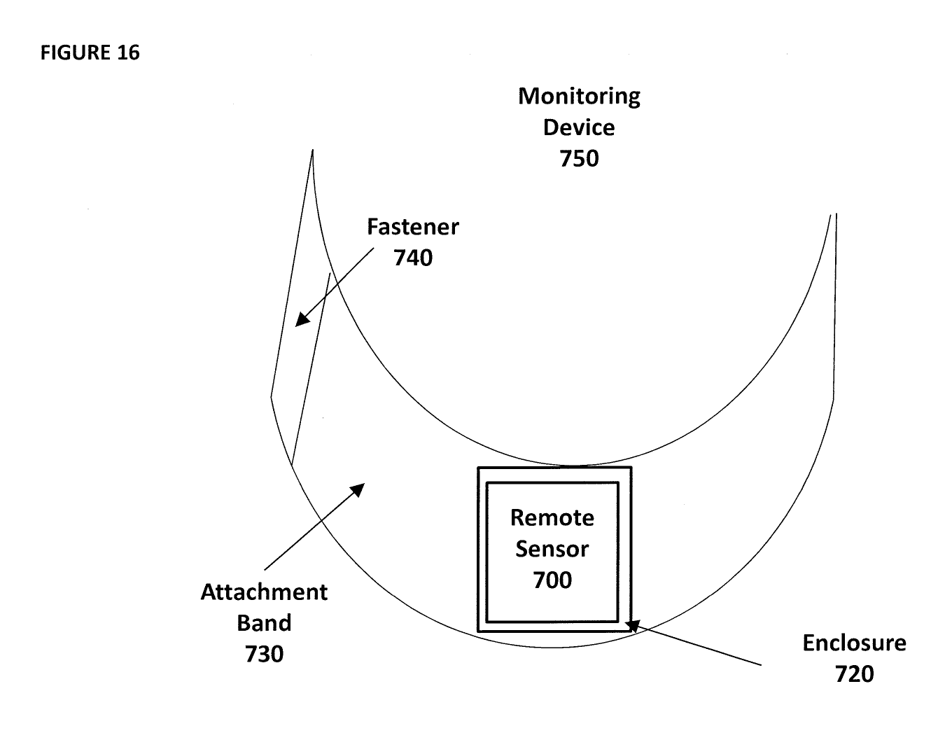

With reference to FIG. 16, an embodiment of monitoring device 750 is depicted. Monitoring device 750 comprises remote sensor 700. Remote sensor 700 is attached to attachment band 730, using known attachment mechanisms such as Velcro, adhesive, stitching, a clear sleeve or covering, etc. Attachment band 730 protects remote sensor 700 but is designed to not interfere with the actions of infrared temperature sensor 620, humidity sensor 630, pressure sensor 640, accelerometer 650, gyroscope 660, magnetometer 670 button 680, and button 690. For example, attachment band 730 optionally comprises an opening in which infrared temperature sensor 620, humidity sensor 630, pressure sensor 640, accelerometer 650, gyroscope 660, magnetometer 670, button 680, and button 690 are placed, and those devices can therefore be placed in direct contact with the patient.

Attachment band 730 is a flexible band that can be attached to a human being, such as a strap that attaches to a wrist, arm, leg, or head. Monitoring device 750 further comprises fastener 740, which also is a known attachment mechanism such as Velcro, a clip, hooks, etc. Fastener 740 can be used to attach one end of attachment band 730 to the other end of attachment band 730 to secure attachment band 730 around a wrist, arm, leg, or head, etc.

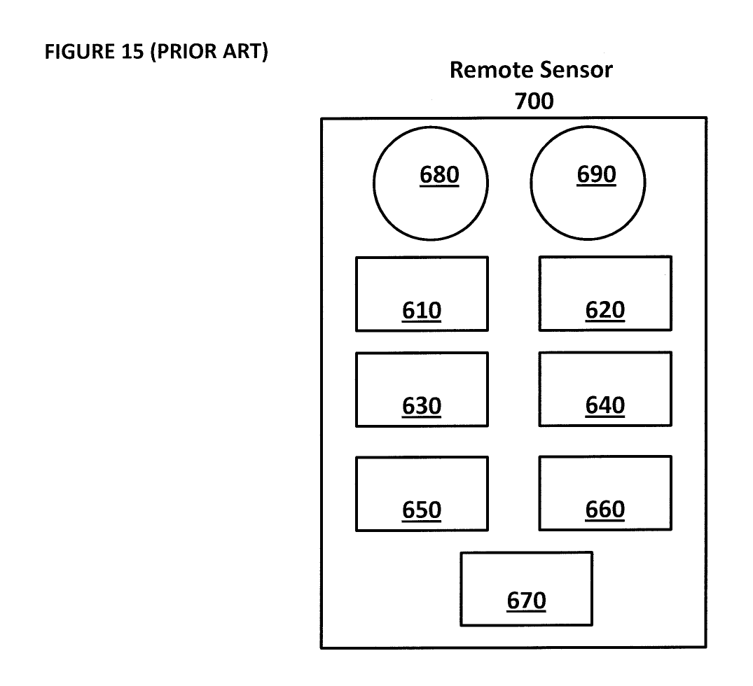

With reference to FIGS. 1 and 15, remote sensor 700 (an example of medical device 10) collects data from a patient, including temperature of the patient measured by infrared temperature sensor 620, humidity of the environment surrounding the patient measured by humidity sensor 630, pressure between remote sensor 700 and the patient measured by pressure sensor 640, acceleration of remote sensor 700 in three directions (e.g., the X, Y, and Z directions) measured by accelerometer 650, the movement of remote sensor 700 in three directions (e.g., the X, Y, and Z directions) measured by gyroscope 660, and the north direction indicated by magnetometer 670. Button 680 and button 690 each can be pressed by the human patient. When this happens, remote sensor 700 generates a signal indicating which button—button 680 or button 690 or both—was pressed.

This data is transmitted by remote sensor 700 to computing device 20 over a Bluetooth wireless connection. As discussed previously with reference to FIG. 1, computing device 20 communicates with server 30 over a network 25. Server 30 is coupled to data store 50. Server 30 is also coupled to computing device 40 over network 25. In this embodiment, computing device 40 is operated by a physician. Server 30 is coupled to computing device 60 over network 25. Computing device 60 is coupled to data store 70.

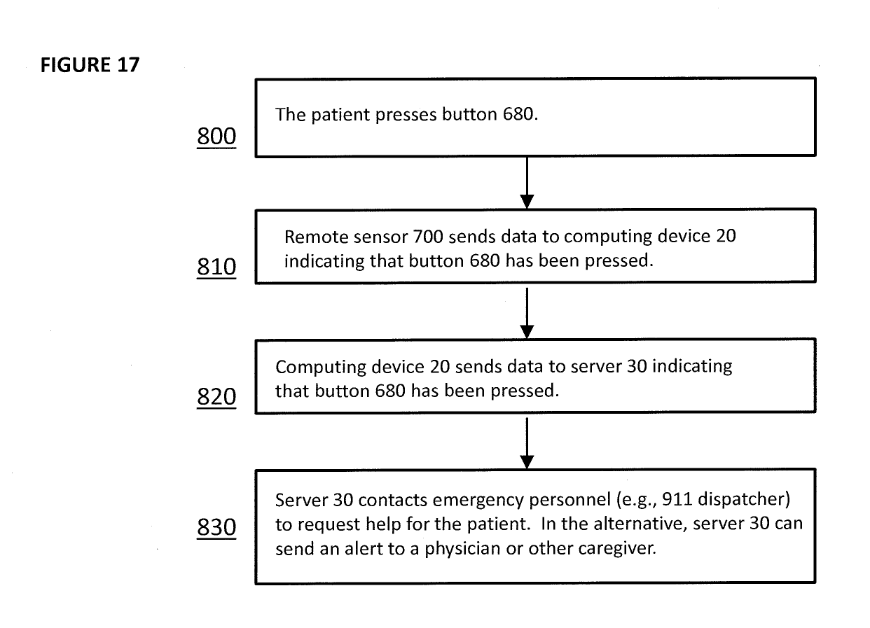

With reference to FIG. 17, an embodiment of a method is shown. Button 680 can be used to indicate an emergency situation. If the patient needs emergency personnel to assist her, the patient presses button 680 (step 800). Remote sensor 700 sends data to computing device 20 indicating that button 680 has been pressed (step 810). Computing device 20 sends data to server 30 indicating that button 680 has been pressed (step 820). Server 30 contacts emergency personnel (e.g., 911 dispatcher) to request help for the patient, or in the alternative, server 30 can send an alert (such as an email, SMS message, or phone call) to a physician or other caregiver (step 830). The alert can be generated by notification engine 120 in response to information from risk classification engine 100 (which classifies the risk based on button 680 being pressed), as discussed previously.

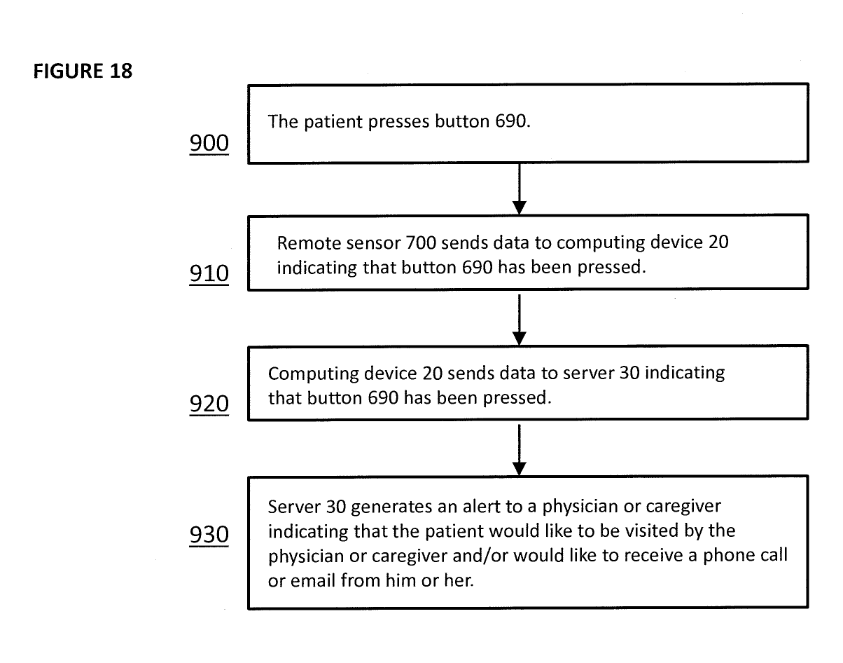

With reference to FIG. 18, an embodiment of another method is shown. Button 690 can be used to indicate a non-emergency situation where the patient wishes to be contacted by his or her physician or caregiver. If the patient would like his or her physician or caregiver to visit him or her or to call him or her on the phone, the patient presses button 690 (step 900). Remote sensor 700 sends data to computing device 20 indicating that button 690 has been pressed (step 910). Computing device 20 sends data to server 30 indicating that button 690 has been pressed (step 920). Server 30 sends an alert (such as an email, SMS message, or phone call) to a physician or caregiver indicating that the patient would like to be visited by the physician or caregiver and/or would like to receive a phone call or email from him or her (step 930). The alert can be generated by notification engine 120 in response to information from risk classification engine 100 (which classifies the risk based on button 690 being pressed), as discussed previously.

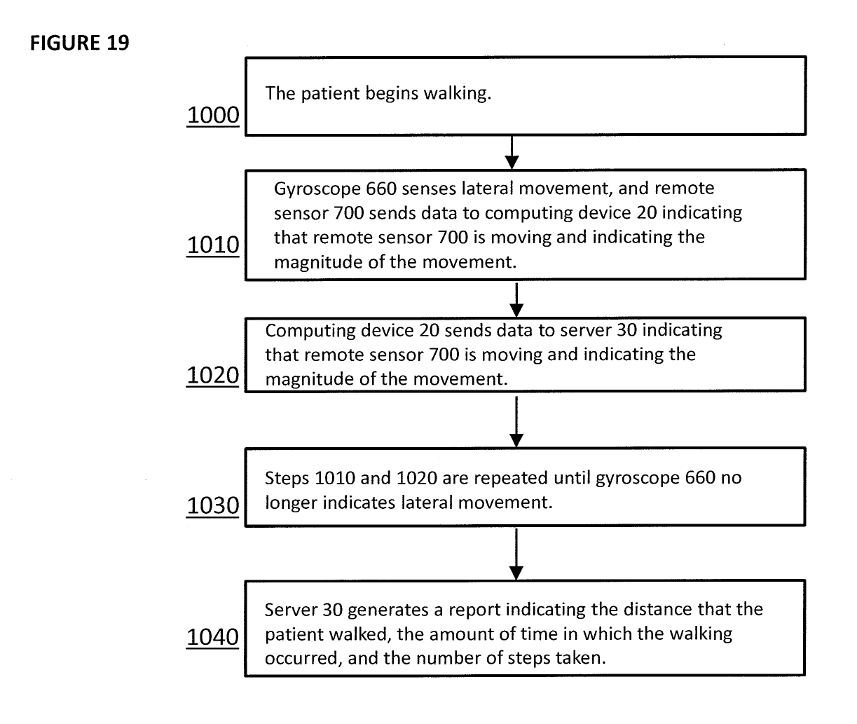

With reference to FIG. 19, an embodiment of another method is shown. The patient wears monitoring device 750 on his or her leg or waist. The patient begins walking (step 1000). Gyroscope 660 detects lateral movement, and remote sensor 700 sends data to computing device 20 indicating that remote sensor 700 is moving and indicating the magnitude of the movement (step 1010). Computing device 20 sends data to server 30 indicating that remote sensor 700 is moving and indicating the magnitude of the movement (step 1020). Steps 1010 and 1020 are repeated until gyroscope 660 no longer indicates lateral movement (step 1030). Server 30 generates a report indicating the distance that the patient walked, the amount of time in which the walking occurred, and the number of steps taken (step 1040). The number of steps taken is determined based on data collected during the calibration process, discussed below.

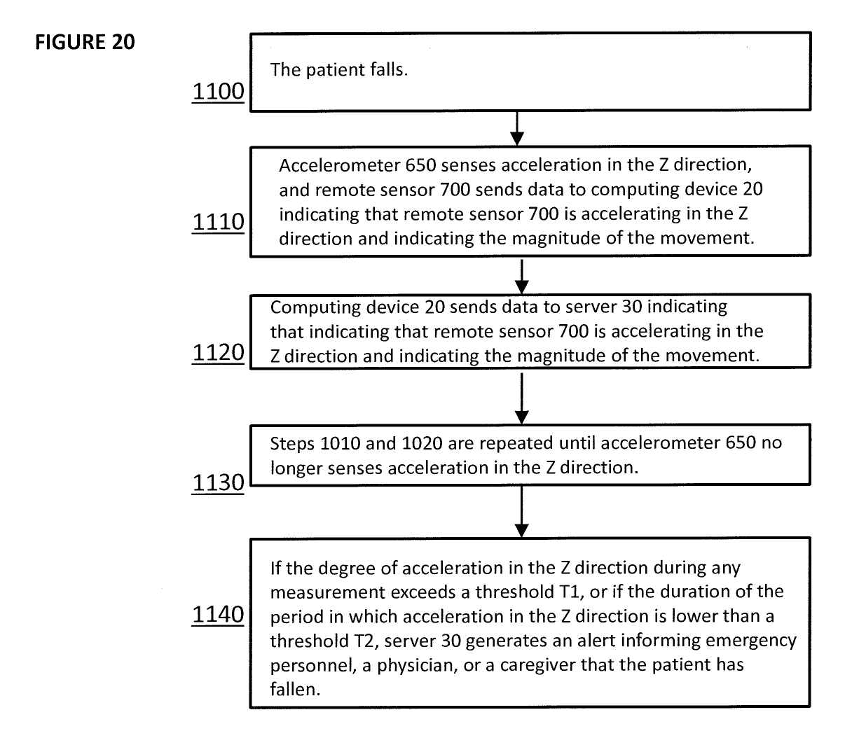

With reference to FIG. 20, an embodiment of another method is shown. The patient wears monitoring device 750 on his or her arm, leg, or hip. The patient falls (step 1100). Accelerometer 650 senses acceleration in the Z direction, and remote sensor 700 sends data to computing device 20 indicating that remote sensor 700 is accelerating in the Z direction and indicating the magnitude of the movement (step 1110). Computing device 20 sends data to server 30 indicating that indicating that remote sensor 700 is accelerating in the Z direction and indicating the magnitude of the movement (step 1120). Steps 1010 and 1020 are repeated until accelerometer 650 no longer senses acceleration in the Z direction (step 1130). If the degree of acceleration in the Z direction during any measurement exceeds a threshold T1, or if the duration of the period in which acceleration in the Z direction is lower than a threshold T2, server 30 generates an alert (such as an email, SMS message, or phone call) informing emergency personnel, a physician, or a caregiver that the patient has fallen (step 1140). The alert can be generated by notification engine 120 in response to information from risk classification engine 100 (which classifies the risk based on data from accelerometer 650), as discussed previously.

In FIG. 20, threshold T1 is a number that typically indicates unintended falling (such as when a patient trips and falls) as opposed to intentional movement (such as when a patient lies down in bed). An example of T1 is 9.0 m/s². Acceleration of this amount or greater usually indicates unintended falling and resultant acceleration due to gravity.

Threshold T2 is a number that typically indicates unintended falling (such as when a patient trips and falls) as opposed to intentional movement (such as when a patient lies down in bed). An example of T2 is 3 seconds. For example, acceleration of 3 seconds or less might indicate that a patient has tripped and fallen to the floor. By contrast, lying down in bed usually requires acceleration for more than 3 seconds.

The numbers provided for T1 and T2 above are mere examples, and other values can be used instead.

Some of the embodiments described above will require the calibration of remote sensor 700. Prior to manufacturing of remote sensor 700, testing of a prototype of remote sensor 700 can be performed with several patients who act as test subjects. Each patient can walk with the remote sensor 700, and the patient or an outside observer (or a pedometer as is known in the prior art) can count the number of steps taken. Remote sensor 700 will record the lateral distance travelled. From this data, one can determine the average step size or lateral distance travelled with each step. This information can then be programmed into remote sensor 700, such that when a patient begins laterally moving, as in FIG. 19, the number of steps taken can be estimated with significant accuracy.

Similarly, the patients can use remote sensor 700 when they lie down in bed. Data can then be collected from each remote sensor 700 to determine information such as the average distance above ground at which a patient will sleep and the average distance that a patient will move toward the ground when the lie down to sleep (i.e., from a standing-up position to a lying down position). This data can be programmed into remote sensor 700 and can be used to help determine whether a patient is sleeping or has fallen and needs emergency medical attention. Measurement of maximum acceleration and duration of acceleration also can be performed to determine appropriate values for T1 and T2.

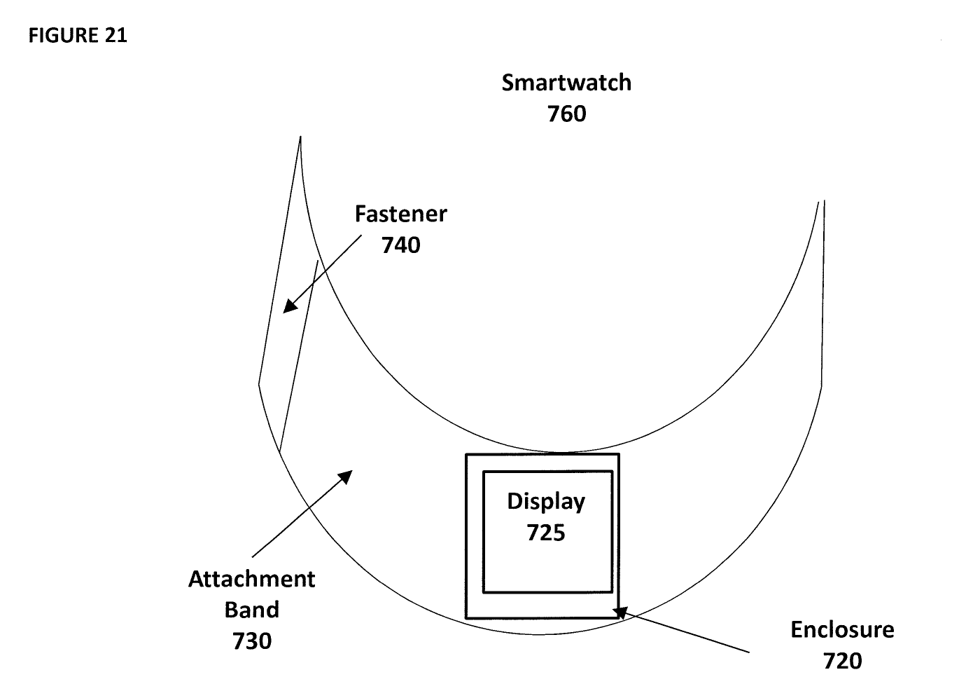

In one embodiment, monitoring device 750 is a smartwatch 760. With reference to FIG. 21, smartwatch 760 comprises enclosure 720, attachment band 730, and fastener 740 as described previously. Smartwatch 760 also comprises display 725, which optionally can be an LCD or LED display.

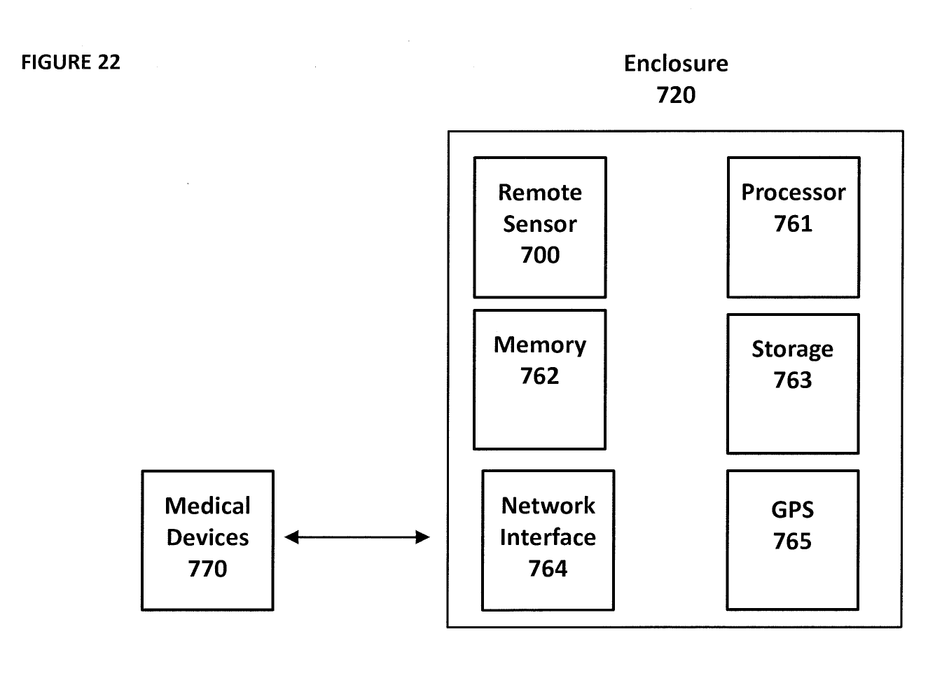

With reference to FIG. 22, enclosure 720 includes remote sensor 700 (described previously), processor 761, memory 762, non-volatile storage 763, network interface 764, and GPS unit 765. Network interface 764 can comprise one or more wired or wireless interfaces, such as an Ethernet interface, WiFi (e.g., 802.11), WiMax, cell phone (e.g., 3G or 4G), Bluetooth, or other interface. As discussed previously, remote sensor 700 comprises a Bluetooth interface. In this embodiment, remote sensor 700 can communicate with another device using its own Bluetooth interface or using network interface 764. Smartwatch 760 comprises an operating system, music and video applications, and other software applications, including a web browser and time keeping application. The timekeeping function can be configured locally or synchronized with a server. Smartwatch 760 can be configured to provide an analog timekeeping display or digital timekeeping display.

Smartwatch 760 optionally can interface with one or more medical devices 770 using network interface 764. Examples of medical devices 770 include blood pressure monitors, weight scales, glucometers, pulse oximeters, electrocardiograph machines, and any other device that can monitor physiological signs and transmit the data wirelessly to network interface 764.

Display 725 can be used to provide user interface screens. The user interface screens can provide user interface functionality to display an on-board sensor menu or external medical device menu. Display 725 also can display medical information, charts, alerts, notifications, etc., as described previously. Smartwatch 760, through network interface 764, can serve as a gateway for medical devices 770, where smartwatch 760 facilitates communication and information transfer between the medical devices 770 and servers and the Internet in general.

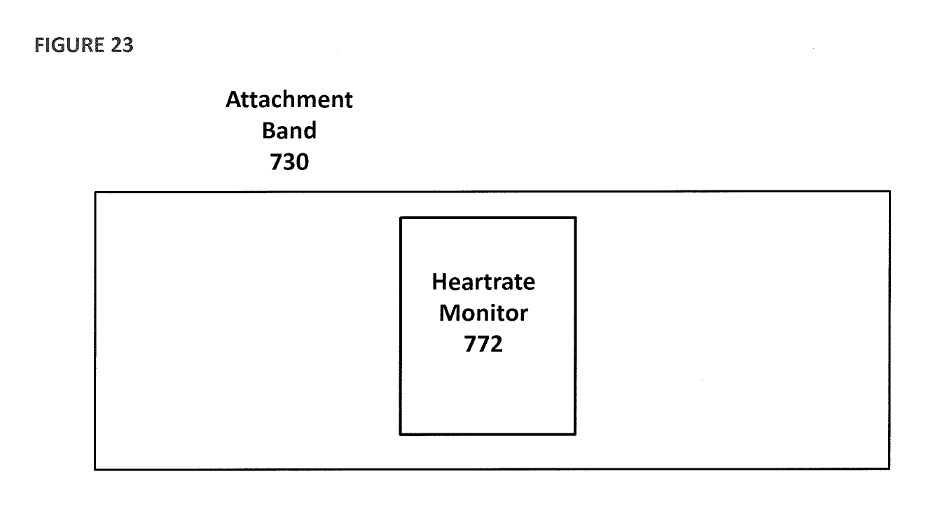

With reference to FIG. 23, smartwatch 760 comprises heart rate monitor 772. Heart rate monitor optionally is attached to the backside of attachment band 730, such that heart rate monitor 772 is in physical contact with the user. Heart rate monitor 772 optionally is a two-lead heart rate monitor known in the prior art. Heart rate monitor 772 can be used to capture the heart rate of the user. That information is accessible to applications operated by smartwatch 760.

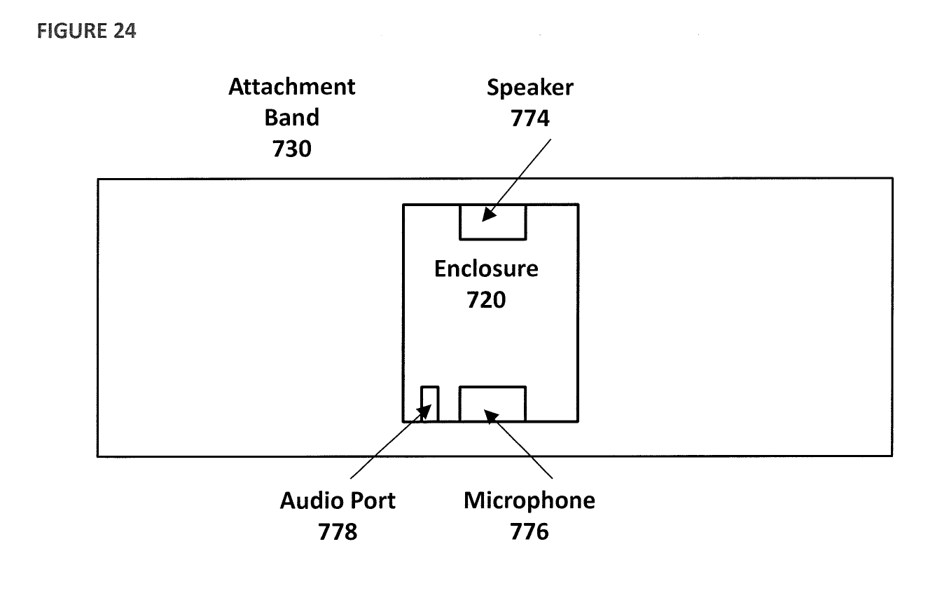

With reference to FIG. 24, smartwatch 760 optionally comprises speaker 774 and microphone 776. Speaker 774 and microphone 776 can be used for voice communication. This feature can be particularly useful for fall detection. Smartwatch 760 optionally comprises audio port 778, which can be used to receive a wired headset or an earphone-microphone apparatus as is known in the prior art.

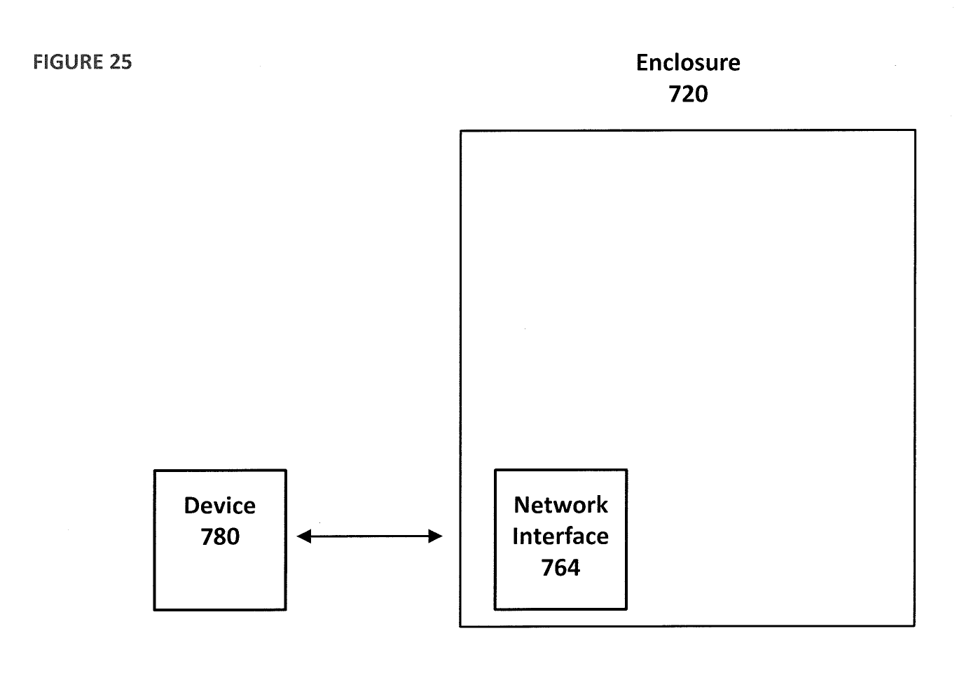

With reference to FIG. 25, smartwatch 760 optionally can serve as a gateway for device 780. Device 780 can be any device with a wireless network interface that can communicate with network interface 764. Device 780 can be a wearable sensor or any appliance (such as a washing machine, refrigerator, toaster, air conditioner unit, heating unit, thermostat, car etc.). Device 780 needs a gateway for access to the Internet, and smartwatch 760 can serve as that gateway.

References to the present invention herein are not intended to limit the scope of any claim or claim term, but instead merely make reference to one or more features that may be covered by one or more of the claims. Materials, processes and numerical examples described above are exemplary only, and should not be deemed to limit the claims. It should be noted that, as used herein, the terms “over” and “on” both inclusively include “directly on” (no intermediate materials, elements or space disposed there between) and “indirectly on” (intermediate materials, elements or space disposed there between). Likewise, the term “adjacent” includes “directly adjacent” (no intermediate materials, elements or space disposed there between) and “indirectly adjacent” (intermediate materials, elements or space disposed there between). For example, forming an element “over a substrate” can include forming the element directly on the substrate with no intermediate materials/elements there between, as well as forming the element indirectly on the substrate with one or more intermediate materials/elements there between.

What is claimed is:

1. A smartwatch for remotely monitoring a patient, comprising:
a processor;
a memory coupled to the processor;
a remote sensor coupled to the processor for capturing movement data, temperature data, and acceleration data; and
a network interface coupled to the processor for communicating with a computing device.

2. The smartwatch of claim 1, wherein the smartwatch provides a timekeeping function.

3. The smartwatch of claim 1, wherein the network interface enables Bluetooth communication with the computing device.

4. The smartwatch of claim 1, wherein the smartwatch is coupled to a medical device using the network interface.

5. The smartwatch of claim 1, further comprising a GPS unit.

6. A method of monitoring a patient wearing a smartwatch, comprising:
detecting, by a gyroscope contained within the smartwatch, lateral movement of a patient;
transmitting data regarding the lateral movement to a computing device over a wireless connection;
transmitting, by the computing device, data regarding the lateral movement to a server;
generating, by the server, a report indicating the distance that the patient has moved.

7. The method of claim 6, wherein the report indicates the amount of time in which the movement occurred.

8. The method of claim 6, wherein the report indicates the number of steps taken by the patient.

9. The method of claim 6, wherein the gyroscope is contained within a remote sensor.

10. The method of claim 6, wherein the report is provided by a web server to a web browser.

11. The method of claim 6, further comprising providing, by the smartwatch, the current time.

12. The method of claim 6, wherein the wireless connection comprises a Bluetooth connection.

13. A method of detecting a fall by a patient wearing a smartwatch, comprising:
sensing, by an accelerometer contained with the smartwatch, acceleration;
transmitting data regarding the acceleration to a computing device;
transmitting, by the computing device, data regarding the acceleration to a server determining, by the server, that the degree of acceleration exceeds a predetermined threshold T1; and
generating, by the server, an alert indicating that the patient has fallen.

14. The method of claim 13, wherein the alert is a phone call.

15. The method of claim 13, wherein the alert is an email.

16. The method of claim 13, wherein the alert is an SMS message.

17. The method of claim 13, further comprising providing, by the smartwatch, the current date and time.

18. The method of claim 13, wherein the step of transmitting data regarding the acceleration to a computing device occurs over a Bluetooth interface.

19. The method of claim 13, wherein the smartwatch comprises a GPS unit.

20. The method of claim 18, further comprising providing, by the smartwatch, the current date and time.
